(19) United States
(12) Patent Application Publication
(10) Pub. No.: US 2003/0040720 A1
(43) Pub. Date: Feb. 27, 2003

(54) Needle hub assembly

(76) Inventor: Gregory Alan Steube, St. Charles, MI (US)

Correspondence Address: Mark S. Leonardo, Esq., Brown Rudnick Freed & Gesmer, One Financial Center, Box IP, 18th Floor, Boston, MA, 02111, US

(21) Appl. No.: 09/935,151

(22) Filed: Aug. 22, 2001

(51) Int. Cl. A61M 5/00

(52) U.S. Cl. 604/240

(57) ABSTRACT

A needle assembly is provided that includes a needle hub having an interior cavity and at least one fin therein, wherein a barrel tip of a syringe barrel engages with at least one fin of the needle hub. The barrel tip may be elongated and extends from a distal end of the barrel. The interior cavity may have a substantially annular configuration. The barrel tip may form a substantial seal adjacent to at least one fin. This configuration advantageously reduces dead space within which medication may be wasted.

### BACKGROUND

1. Technical Field

The present disclosure generally relates to the field of medical needle assemblies for the administration of fluids, and more particularly, to a needle hub assembly that minimizes fluid waste.

2. Description of the Related Art

Medical needle assemblies, such as, for example, syringes are well known for the administration of fluid injections, such as, for example, medication, etc. Existing syringe products include permanent needle syringes, luered fitting syringes, etc. Typically, a luer fitting includes a tapered conical nozzle at a distal end of a syringe barrel. Luer fittings may include a threaded collar for securing a needle hub assembly to the nozzle.

For example, a prior art syringe 10, as shown in FIG. 1, includes a needle hub 12 connected to a syringe barrel 14. Needle hub 12 has a tubular section 16 that backfills with medication for an injection through a needle cannula 18. Tubular section 16 is designed to reduce the amount of dead space found in a needle hub and thus reduces the amount of medication wasted upon delivery of a drug through needle 18. A luer tip 20 at the bottom of syringe barrel 14 sealingly engages tubular section 16 due to the luer taper of tip 20. Often, overtightening or undertightening of needle hub 12 to barrel 14 can cause a nozzle 22 of tubular section 16 to be displaced overly forward or rearward for engagement with a plunger 24 in barrel 14 at 26. This disadvantageously creates dead space, trapping medication at 28 and 30 and thus not optimizing the reduction in dead space for needle hub 12.

The term “dead space” refers to the space created by the mating recess between a needle assembly and a conical fitting of a luer. In luer based syringes that rely on the luer taper for seating, a minor change in the fitting of the two parts may cause a large increase in dead space. Conventional hypodermic needle hubs waste expensive medication due to the dead space associated with the connection of the needle and syringe. Significant amounts of medication can be trapped in the mating recess following an injection. It is contemplated that 0.08 milliliters of medication can be trapped in the mating recess. For a 1.00 milliliter injection, 8% of the medication is wasted. It is further contemplated that the quantity of medication wasted may be as much as 15%. A significant portion of the cost of delivering an injection is typically the medication expense. Reducing medication waste would significantly reduce healthcare costs.

Attempts have been made to reduce dead space via a tapered sealing means. See, for example, U.S. Pat. Nos. 5,782,803, 5,902,271 and 5,902,277. However, these type devices may have specialized tooling requirements resulting in higher production costs. Further, these devices do not address the drawback of over and under tightening of a needle hub.

Therefore, it would be desirable to overcome the disadvantages of the prior art with a needle hub assembly that employs an engagement surface to reduce the amount of wasted medication.

### SUMMARY

Accordingly, a needle hub assembly having an engagement surface is disclosed to reduce the amount of wasted medication. In one particular embodiment, a needle assembly is provided, in accordance with the principles of the present disclosure. The needle assembly includes a needle hub having an interior cavity and at least one fin disposed therein. A barrel has a barrel tip which is disposed within the interior cavity and engages the at least one fin of the needle hub. The barrel tip may be elongated and extend from a distal end of the barrel. The interior cavity may have a substantially annular configuration. The barrel tip may form a substantial seal adjacent to the at least one fin. This configuration advantageously reduces dead space associated with the connection between the needle hub and the syringe barrel.

In an alternate embodiment, the needle hub has a needle support defining an interior cavity about at least a portion thereof. The interior cavity has at least one fin formed therein. The needle assembly also includes a barrel having a proximal end and distal end. The distal end supporting the needle hub and including an elongated barrel tip which is received within the interior cavity of the needle hub. The barrel tip engaging the at least one fin. The barrel tip may form a substantial seal with the needle support. Desirably, the barrel tip forms a substantial seal with the needle support adjacent to the at least one fin. The interior cavity of the needle hub may have a plurality of fins formed therein. Desirably, the interior cavity of the needle hub may have four fins formed therein.

The needle hub may include a hub skirt mounted to the distal end of the barrel. The interior cavity of the needle hub may be defined between the hub skirt and the needle support. The needle support may define a needle cavity having at least a portion of a needle cannula disposed therein. The interior cavity of the needle hub may be coaxial with the needle cavity. The interior cavity of the needle hub and the needle cavity may be in substantially parallel alignment.

The barrel of the syringe may be configured to receive a plunger. The plunger may be configured to engage a proximal opening of the needle support. The needle cannula may have at least a portion disposed within the needle support adjacent to a proximal end thereof.

### BRIEF DESCRIPTION OF THE DRAWINGS

The objects and features of the present disclosure, which are believed to be novel, are set forth with particularity in the appended claims. The present disclosure, both as to its organization and manner of operation, together with further objectives and advantages, may be best understood by reference to the following description, taken in connection with the accompanying drawings wherein:

FIG. 1 is a cross-sectional view of a prior art needle hub assembly;

FIG. 2 is a cross-sectional view of a needle assembly, in accordance with the principles of the present disclosure;

FIG. 3 is a cross-sectional view of an alternate embodiment of a needle hub of the needle assembly shown in FIG. 2;

FIG. 4 is a bottom view of the needle assembly shown in FIG. 2;

FIG. 5 is a cross-sectional view of the needle hub shown in FIG. 2 taken along lines A-A shown in FIG. 4; and

FIG. 6 is a cross-sectional view of the needle hub shown in FIG. 2 taken along lines B-B shown in FIG. 4.

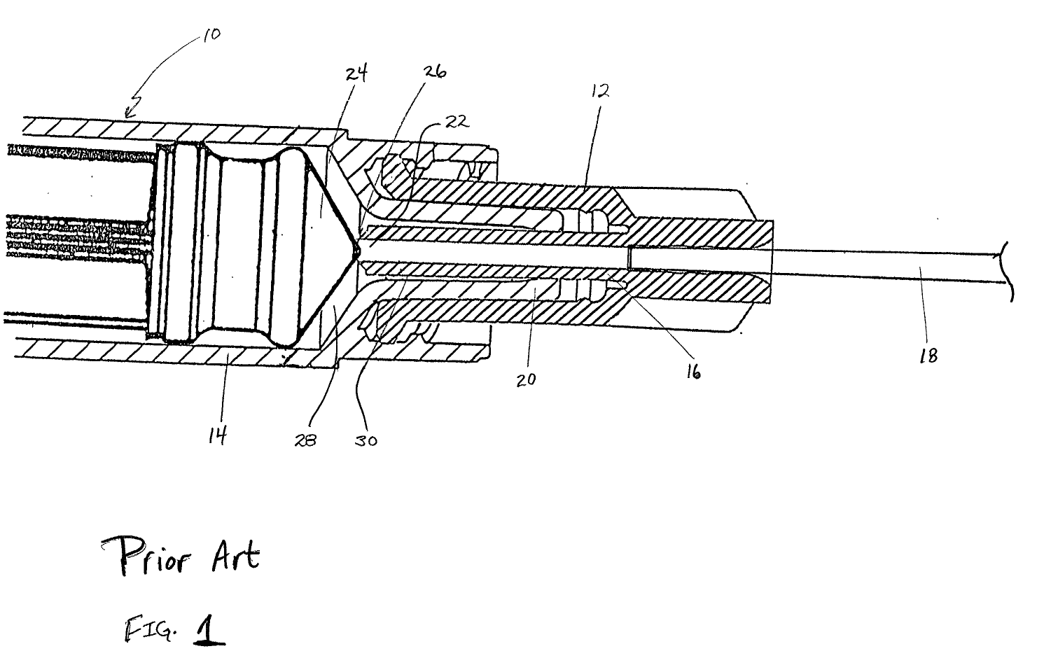

### DETAILED DESCRIPTION OF EXEMPLARY EMBODIMENTS

The exemplary embodiments of the needle assembly and methods of operation disclosed are discussed in terms of administration of fluids to and/or from a subject, and more particularly, in terms of needle hub assemblies that minimize waste of medication during an injection. It is envisioned that the present disclosure finds application to the injection of preventive medications, medicaments, etc., as well as injections employed during procedures relating to phlebotomy, dental, orthopedic, digestive, intestinal, urinary, veterinary types, etc., to a subject.

In the discussion which follows, the term “proximal” will refer to the portion of a structure which is closer to the practitioner, while the term “distal” will refer to the portion which is further from the practitioner. As used herein, the term “subject” refers to a patient which receives injections from a syringe. According to the present disclosure, the term “practitioner” refers to an individual administering an injection, installing or removing a needle hub assembly to or from a syringe, and may include support personnel.

The component parts of the needle assembly are fabricated from materials suitable for medication injections, such as, for example, polymerics or metals, such as stainless steel, depending on the particular medical needle application and/or preference of a practitioner. Semi-rigid and rigid polymerics are contemplated for fabrication, as well as resilient materials, such as molded medical grade polypropylene. One skilled in the art, however, will realize that other materials and fabrication methods suitable for assembly and manufacture, in accordance with the present disclosure, also would be appropriate.

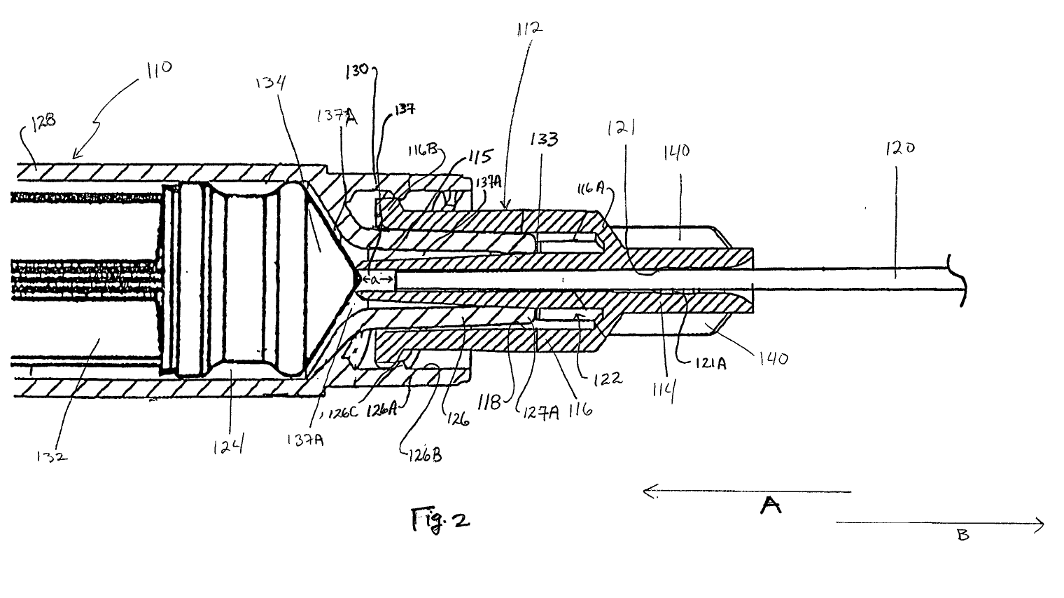

Reference will be now be made in detail to the exemplary embodiments of the disclosure, which are illustrated in the accompanying figures. Turning now to the figures wherein like components are designated by like reference numerals throughout the several views and initially to FIG. 2, there is illustrated a needle assembly, such as, for example, a syringe 110, in accordance with the principles of the present disclosure.

Syringe 110 includes a needle hub 112 and a barrel 124. Barrel 124 engages an engagement surface disposed within an interior cavity of needle hub 112, as will be discussed, to advantageously reduce the amount of medication wasted during administration of an injection to a subject.

Needle hub 112 has a needle support 114, a hub skirt 116 and an interior cavity 118 defined therebetween. Hub skirt 116 projects outwardly along a web portion 116A and extends proximally along the longitudinal length of needle hub 112 to a flange 116B. Flange 116B facilitates mounting needle hub 112 to barrel 124, as will be described, and provides stability during operation of syringe 110.

Hub skirt 116 cooperates with needle support 114 to define a substantially annular configuration of interior cavity 118. Interior cavity 118 may alternately be configured, such as, for example, by length, width, etc., according to the requirements for a particular medical needle application and/or preference of a practitioner. Interior cavity 118 may also have various geometric configurations, such as, for example, rectangular cross-section, intermittent cavities, undulating, etc., depending on, for example, strength, flexibility, etc.

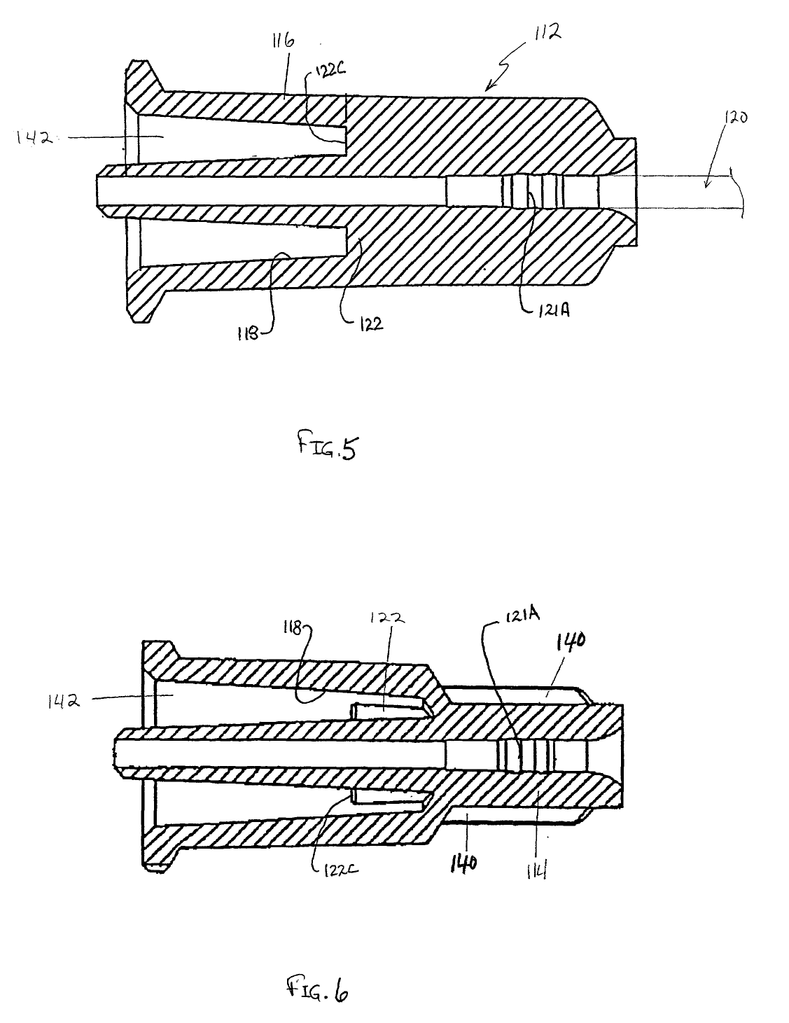

Needle support 114 defines a needle cavity 121 that extends to a nozzle 115. Needle cavity 121 is coaxial with interior cavity 118. Alternatively, needle cavity 121 may be offset, concentric, etc., from interior cavity 118. A needle cannula 120 is disposed within needle cavity 121 and extends through a distal end of needle support 114. Beads 121A (also shown in FIGS. 5 & 6) engage and grip needle cannula 120 to facilitate maintenance and proper positioning within needle cavity 121. It is contemplated that needle cannula 120 may be mounted in needle cavity 121 by any suitable means, such as, for example, press fit, friction fit, adhesive, etc. Needle cannula 120 may alternatively be monolithically formed with needle support 114.

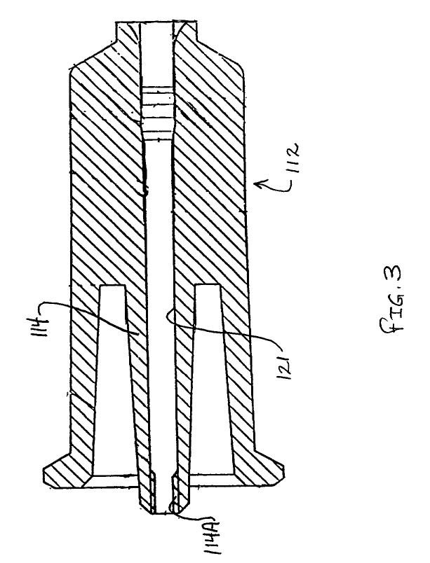

Needle cannula 120 is mounted within needle cavity 121 such that a proximal end of needle cannula 120 is disposed adjacent the proximal end of needle support 114. The proximal end of needle cannula 120 is recessed distally, a distance a, from the proximal end of needle support 114. Thus, the smaller inner diameter of needle cannula 120, relative to the inner diameter of needle cavity 121, reduces the volume of the fluid pathway of syringe 110, including space 137. One of the advantages of this configuration is the reduction of total dead space, which may include space 137, typically created due to a barrel and needle hub connection. Alternatively, as shown in FIG. 3, needle hub 112 includes a cannula stop 114A formed at a proximal end of needle support 114. Cannula stop 114A projects into needle cavity 121 and is disposed about an inner circumference of needle support 114. Cannula stop 114A prevents needle cannula 120 (FIG. 2) from extending beyond the proximal end of needle support 114.

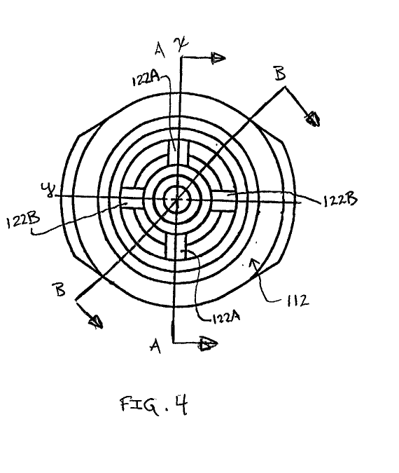

Referring to FIGS. 4-6, fins 122 are formed within a distal portion of interior cavity 118. Fins 122 have a transverse cross-sectional configuration in that two pair of fins 122 intersect. A first pair of parallel fins 122A lie in the same plane x. This plane is perpendicular to a plane y of which the remaining pair of parallel fins 122B lie, resembling a cross configuration. Each of fins 122 extend proximally from a distal end of interior cavity 118 to an engagement surface 122C. Engagement surfaces 122C are substantially planar for engaging a distal end of barrel 124 (FIG. 2), discussed below. One of the advantages of this configuration is that a positive stop is provided for engagement between needle hub 112 and barrel 124 and correspondingly assembly of syringe 110. This reduces dead space associated with the connection of needle hub 112 and barrel 124 by precisely positioning the component parts of syringe 110. It is envisioned that engagement surface 122C may have non-planar configurations, such as, for example, angular, convex, concave, etc., according to the requirements of a particular medical needle application.

It is not required that fins 122 form parallel pairs, as they may be offset. It is contemplated that fins 122 may be relatively disposed at various angles. Fins 122 may be monolithically formed with/or integrally connected to needle support 114 within interior cavity 118 of needle hub 112. It is further contemplated that fins 122 may be attached to needle support 114 by adhesive, clips, pins, etc. It is envisioned that fins 122 may be disposed at various positions along the longitudinal length of interior cavity 118 to provide a positive stop feature for engagement with barrel 124, according to the particular requirements of a medical needle application and/or preference of a practitioner.

Referring back to FIG. 2, barrel 124 has a proximal end 128 and a distal end 130. Distal end 130 of barrel 124 includes an elongated barrel tip 126, a collar 126A and a cavity 126B defined therebetween, configured for receipt of hub skirt 116. Collar 126A includes a bead 126C formed about an inner surface thereof to releasably retain flange 116B within cavity 126B. Flange 116B is snap fit over bead 126C. Bead 126C and/or collar 126A may elastically deform upon engagement with flange 116B for retaining needle hub 112 with barrel 124. It is contemplated that needle hub 112 may be threaded with barrel 124 or, alternatively, may be permanently attached via adhesive, etc.

Barrel tip 126 extends distally from distal end 130 for mounting within interior cavity 118 of needle hub 112. During assembly, hub skirt 116 is mounted to distal end 130 of barrel 124, in the direction shown by arrow A, such that barrel tip 126 is received within interior cavity 118, in the direction shown by arrow B. Barrel tip 126 tapers inwardly to a tip end 127A. Tip end 127A is configured to engage fins 122 and form a substantial seal 133 with needle support 114 adjacent to fins 122. Substantial seal 133 may include formation via friction fit, snap-lock etc. Tip end 127A engages fins 122 in a positive stop configuration to advantageously reduce potential dead space 137A associated with the connection of needle hub 112 and barrel 124, as discussed. Engagement of barrel tip 126 may also form a substantial seal with other surfaces defining interior cavity 118.

Proximal end 128 is configured for slidable receipt and support of a plunger 132 which includes an elastomearic tip 134 at its distal end. Tip 134 is configured to engage nozzle 115 of needle support 114 to force medication through needle cannula during an injection.

Although shown as a manually manipulated syringe 110, it is contemplated that movement of plunger 128 relative to barrel 124 may be controlled through motorized mechanisms, electronic components, etc.

Needle hub 112 includes four projections 140 disposed about the exterior surface of needle support 114. Projections 140 facilitate manipulation of needle hub 112 for assembly with barrel 124. Further, projections 140 facilitate manipulation of syringe 110 during a medical needle procedure. Projections 140 may be co-linear with fins 122. It is envisioned that needle hub 112 may have a plurality of projections or no projections disposed about the exterior surface of needle support 114.

The assembly and operation of syringe 110, similar to that illustrated above, will now be described. Initially, needle hub 112 is manipulated via projections 140. Hub skirt 116 is caused to engage the distal end of barrel 124, in the direction shown by arrow A, such that flange 116B is retained by bead 126C to releasably retain needle hub 112 with barrel 124, as discussed. Barrel tip 126 is correspondingly caused to enter interior cavity 118, in the direction shown by arrow B. Tip end 127A engages fins 122 in a positive stop configuration. Tip end 127A forms a substantial seal 133 with needle support 114 adjacent fins 122. As a result of this configuration, needle hub 112 is attached to barrel 124 and under and over tightening of the connection is avoided. This reduces dead spaces 137 and 137A associated with the connection. Syringe 110 is prepared for an injection and a practitioner administers a medical needle procedure, such as, for example, a medication injection to a subject.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A needle hub assembly comprising:
a needle hub defining an interior cavity and at least one fin being disposed therein, wherein,
the at least one fin engages a barrel tip when disposed within the interior cavity.

2. A needle hub assembly as recited in claim 1, wherein the barrel tip is elongated and extends from a distal end of a barrel.

3. A needle hub assembly as recited in claim 1, wherein the interior cavity has a substantially annular configuration.

4. A needle hub assembly as recited in claim 1, wherein the barrel tip forms a substantial seal adjacent the at least one fin.

5. A needle assembly comprising:
a needle hub including a needle support and defining an interior cavity about at least a portion of the needle support, the interior cavity having at least one fin formed therein; and
a barrel having a proximal end and a distal end, the distal end supporting the needle hub and including an elongated barrel tip extending therefrom, the barrel tip being received within the interior cavity of the needle hub and engaging the at least one fin.

6. A needle assembly as recited in claim 5, wherein the barrel tip forms a substantial seal with the needle support.

7. A needle assembly as recited in claim 5, wherein the barrel tip forms a substantial seal with the needle support adjacent the at least one fin.

8. A needle assembly as recited in claim 5, wherein the interior cavity of the needle hub has a plurality of fins formed therein.

9. A needle assembly as recited in claim 5, wherein the interior cavity of the needle hub has four fins formed therein.

10. A needle assembly as recited in claim 5, wherein the needle hub includes a hub skirt mounted to the distal end of the barrel.

11. A needle assembly as recited in claim 10, wherein the interior cavity is defined between the hub skirt and the needle support.

12. A needle assembly as recited in claim 5, wherein the needle support defines a needle cavity having at least a portion of a needle cannula disposed therein.

13. A needle assembly as recited in claim 12, wherein the interior cavity and the needle cavity are coaxial.

14. A needle assembly as recited in claim 12, wherein the interior cavity and the needle cavity are in substantially parallel alignment.

15. A needle assembly as recited in claim 5, wherein the barrel is configured for receipt of a plunger.

16. A needle assembly as recited in claim 15, wherein the plunger is configured to engage a proximal opening of the needle support.

17. A needle assembly as recited in claim 5, wherein at least a portion of the needle cannula is disposed with the needle support adjacent a proximal end thereof.

18. A needle assembly comprising:
a barrel including a barrel tip; and
a needle hub means for engaging the barrel tip.

19. A needle hub comprising:
a needle support, a hub skirt and an annular interior cavity defined therebetween, the needle support defining a needle cavity in coaxial alignment with the interior cavity, the needle cavity having a needle cannula disposed therein and having at least a portion thereof disposed adjacent a proximal end of the needle support, the interior cavity having four fins formed at a distal portion thereof.

20. A syringe comprising:
a needle support, a hub skirt and an annular interior cavity defined therebetween, the needle support defining a needle cavity in coaxial alignment with the interior cavity, the needle cavity having a needle cannula disposed therein and having at least a portion thereof disposed adjacent a proximal end of the needle support, the interior cavity having four fins formed at a distal portion thereof;
a barrel having a proximal end and a distal end, the hub skirt being mounted to the distal end of the barrel and the distal end including an elongated barrel tip, the barrel tip being received within the interior cavity, the barrel tip engaging the four fins and forming a substantial seal with the needle hub adjacent the four fins; and
a plunger being slidably received by the barrel and having a distal end configured to engage the proximal end of the needle support.

21. A needle hub defining an interior cavity and having at least one fin, the at least one fin having an engagement surface disposed within the interior cavity.

22. A needle hub as recited in claim 21, wherein the interior cavity has a substantially annular configuration.

23. A needle hub as recited in claim 21, further comprising a barrel having a distal end and a hub skirt, wherein the hub skirt is mounted to the distal end of the barrel.

24. A needle hub as recited in claim 23, further comprising a needle support, wherein the interior cavity is defined between the hub skirt and the needle support.

25. A needle hub as recited in claim 24, wherein the needle support defines a needle cavity having at least a portion of a needle cannula disposed therein.

26. A needle hub as recited in claim 25, wherein the interior cavity and the needle cavity are coaxial.
